(19) United States
(12) Patent Application Publication
(10) Pub. No.: US 2018/0132458 A1
(43) Pub. Date: May 17, 2018

(54) TRANSGENIC ANIMAL MODELS FOR CYSTIC FIBROSIS

(71) Applicants: THE REGENTS OF THE UNIVERSITY OF MICHIGAN, ANN ARBOR, MI (US); WAYNE STATE UNIVERSITY, DETROIT, MI (US)

(72) Inventors: YUQING EUGENE CHEN, ANN ARBOR, MI (US); JIFENG ZHANG, ANN ARBOR, MI (US); JIE XU, FAIRFAX, VA (US); FEI SUN, CANTON, MI (US)

(21) Appl. No.: 15/815,390

(22) Filed: Nov. 16, 2017

Related U.S. Application Data
(60) Provisional application No. 62/423,447, filed on Nov. 17, 2016.

(51) Int. Cl. A01K 67/027; A61K 49/00

(52) U.S. Cl. CPC A01K 67/0276; A61K 49/0008; A01K 2217/206; A01K 2217/054; A01K 2267/0306; A01K 2227/107

(57) ABSTRACT

This disclosure relates to transgenic rabbit models of cystic fibrosis, and methods of using these rabbits and their derivatives.

The present application claims priority to U.S. Provisional Patent Application Ser. No. 62/423,447, filed Nov. 17, 2016, the disclosure of which is herein incorporated by reference in its entirety.

### STATEMENT REGARDING FEDERALLY SPONSORED RESEARCH OR DEVELOPMENT

This invention was made with government support under HL133162, OD020187, and HL096800 awarded by the National Institutes of Health. The Government has certain rights in the invention.

### FIELD OF THE DISCLOSURE

This disclosure relates to transgenic rabbit models of cystic fibrosis, and methods of using these rabbits and their derivatives.

### BACKGROUND OF THE DISCLOSURE

Cystic fibrosis (CF) is the most common fatal autosomal recessive disorder with a disease frequency of 1 in 2,000 live births and a carrier rate of approximately 5% in Caucasian population (Pilewski J M & Frizzell R A (1999) Physiological reviews 79(1 Suppl):S215-255). The disease can be characterized as a malfunction of exocrine tissues in which abnormal regulation of epithelial Cl— channel is associated with pathophysiology of the disease (Welsh M J & Ramsey B W (1998) American journal of respiratory and critical care medicine 157(4 Pt 2):S148-154). The major clinical symptoms and signs include chronic pulmonary disease, pancreatic exocrine insufficiency, intestinal disease and an increase in the concentration of sweat chloride (Wang et al., supra).

Mutations of the gene encoding cystic fibrosis transmembrane conductance regulator (CFTR) lead to CF (Wang et al., supra). The CFTR protein is a multi-domain integral membrane glycoprotein, which functions as a regulator of Cl and other ions transport. More than 2,000 mutations have been identified in the CFTR gene and the number is still growing. An up to date CFTR mutation list is available at the CFTR2 database. The most common mutation in CF is the deletion of a phenylalanine residue at position 508 (F508del), which occurs in more than 70% homozygous CF patients. Accordingly, most research of CF has been focused on F508del.

CF mouse models have made significant contributions towards understanding of the disease and the development of therapies (Wang et al., supra; Grubb B R & Boucher R C (1999) Physiological reviews 79(1 Suppl):S193-214; Keiser N W & Engelhardt J F (2011) Current opinion in pulmonary medicine 17(6):478-483) however, there are notable limitations in translating the information gained from CF mice to the humans. For example, unlike human CF patients, CF mice show a lack pulmonary pathophysiology, absence of obvious pancreatic pathology, and no male infertility problems. A recent report in CFTR knockout (KO) rats revealed that CF rats recapitulated some of the clinic presentations observed in human CF patients. The enthusiasm of using CF rat model is dramatically reduced because these rats don't develop spontaneous lung infections, which is a major cause of CF patient's mortality. In addition, there is no F508del rat model available. CFTR KO ferrets (Sun X, et al. (2010) The Journal of clinical investigation 120(9):3149-3160), and CFTR KO and F508del pigs (Rogers C S, et al. (2008) Science 321(5897):1837-1841; Rogers C S, et al. (2008) The Journal of clinical investigation 118(4):1571-1577), were generated by nuclear transfer. These large animal models have been shown to more closely mimic conditions observed in human CF patients, including lung, pancreatic and liver phenotypes that were not often found in CF mice. However, neither pig nor ferret is a convenient laboratory species. Both CF ferret and pig models are associated with high maintaining cost and required specialized handling skills. These factors have limited the applicability of CF pigs and ferrets almost exclusively to the laboratories originally produced these animals and a few closely associated with them.

### SUMMARY OF THE DISCLOSURE

This disclosure relates to transgenic rabbit models of cystic fibrosis, and methods of using these rabbits and their derivatives.

Experiments described herein on CFTR null rabbits show that CF rabbits suffer from growth impairment, gastrointestinal tract obstruction, micro-gallbladder, and most importantly spontaneous lung infections, and mucus obstructions in their airways. These observations support the CF rabbit described herein as a good animal model, especially because CF rabbits have a similar pathology in the airways to that in human CF patients.

These facts support the use of CF rabbits as major models for CF research as well as drug and therapeutic development. The CF rabbits can be used as novel research tools for the study of (a) an electrophysiological phenotype like that of human cystic fibrosis, (b) pancreatic insufficiency or abnormalities, (c) hepatic abnormalities, (d) gall bladder and/or bile duct abnormalities, (e) sweat gland abnormalities, (f) kidney abnormalities, (g) cystic fibrosis related diabetes, (i) bilateral congenital absence of the vas deferens leading to male infertility, (j) tracheal abnormalities, (k) cystic fibrosis lung disease, (l) cystic fibrosis eye disease, and other aspects associated with CF and CFTR gene and its products. The CF rabbits can also serve as preclinical animal model for validating whether a candidate therapeutic approach can be used in the treatment of cystic fibrosis. These methods involve carrying out a therapeutic approach (e.g., administering a candidate therapeutic agent) to a transgenic rabbit, and monitoring the rabbit for a symptom of cystic fibrosis. Detection of improvement in a symptom of cystic fibrosis indicates the identification of a therapeutic approach (e.g., a candidate therapeutic agent, such as a candidate compound) that can be used in the treatment of cystic fibrosis.

The disclosure provides rabbit models of human cystic fibrosis (CF). The transgenic rabbits carry a genome that has one or more mutations in one or both alleles of the rabbit cystic fibrosis transmembrane regulator (CFTR) gene or a relevant gene(s) resulting in altered rabbit CFTR expression and one or more symptoms of CF in the rabbits.

In certain examples, the CFTR alleles of the genomes of rabbits have been knocked out or mutated resulting in loss of CFTR function, and subsequently result in one or more CF phenotypes in the transgenic rabbits. In one example, the mutation includes an insertion of an exogenous nucleic acid molecule and/or a transcription termination sequence. In another example, the mutation substantially eliminates expression or functions of the endogenous CFTR proteins in cells in which such expression and/or function normally exists, absent the mutation. In the case of a rabbit with a mutation or mutations in both alleles of the CFTR gene, the mutation or mutations in each allele can be identical to one another or can be different.

In certain examples, mutation(s) is introduced to the endogenous CFTR gene of the rabbit genome, such as a three-base pair deletion that leads to deletion of F508 (e.g. F508del mutation) in the expressed CFTR protein. Examples of CFTR mutations that find use in the animals (and cells) of the disclosure include, but are not limited to, (i) class I mutations, which result in little or no mRNA production, and thus little or no protein production (e.g., nonsense mutation (e.g. G542X), a frameshift mutation (e.g., 394delTT), a splice junction mutation (e.g., 1717-1 GtoA), (ii) class II mutations, which result in a protein trafficking defect where CFTR is made, but fails to traffic to the cell membrane (e.g., F508del), (iii) class III mutations, which result in CFTR trafficking to the cell membrane, but failing to be properly regulated or responding to cAMP stimulation (e.g., G551D, which fails to respond to cAMP stimulation), (iv) class IV mutations, which result in a CFTR channel function defect (e.g., R117H), and (v) class V mutations, which cause CFTR synthesis defects, resulting in reduced synthesis or defective processing of normal CFTR (e.g., missense mutation (e.g., A455E), or a mutation introduced by alternative splicing. Additional mutations include W1282X, R347P, R553X, and N1303K. In all cases, the mutation(s) lead to loss or reduced CFTR function, and one or more CF phenotypes in the transgenic rabbits.

The transgenic rabbit models of the disclosure may optionally include a transgenic copy of a wild-type or mutated gene in the form of cDNA or genomic DNA from a different animal. For example, a rabbit in which an endogenous CFTR gene is mutated or inactivated may be modified to include a CFTR gene from another animal (such as a human), which may be wild-type or may include a mutation (e.g., F508del CFTR). In one example, a rabbit with its endogenous rabbit CFTR knocked-out expresses a human transgene encoding a mutant CFTR gene, such as the F508del CFTR gene. In another example, a wild-type human CFTR gene under the control of an intestinal cell-specific promoter (e.g. intestinal fatty acid binding protein (iFABP)) is introduced to the rabbit genome whose endogenous CFTR gene is mutated or inactivated, to alleviate CF phenotype in the intestines.

As described further herein, the rabbits of the disclosure can have one or more phenotypes including, but not limited to, (a) an electrophysiological phenotype like that of human cystic fibrosis, (b) pancreatic insufficiency or abnormalities, (c) hepatic abnormalities, (d) gall bladder and/or bile duct abnormalities, (e) sweat gland abnormalities, (f) kidney abnormalities, (g) cystic fibrosis related diabetes, (i) bilateral congenital absence of the vas deferens leading to male infertility, (j) tracheal abnormalities, (k) cystic fibrosis lung disease, and (l) cystic fibrosis eye problems.

The rabbits of the disclosure provide several advantages, as they represent clinically relevant non-human animal models of CF that find use in understanding the pathogenesis of CF, in the identification, development and validation of therapies for this devastating disease. Despite many progresses in understanding and treating CF, the pathogenesis of the disease is not fully understood and therapies remain be improved. Availability of CF rabbits allows investigators to address key basic problems that have persisted unresolved for years, and facilitates preclinical drug discovery work for identifying therapeutic agents that can be used in the treatment of cystic fibrosis.

Additional embodiments are described herein.

### DESCRIPTION OF THE FIGURES

FIG. 1 shows Panel A) CFTR null rabbits (“−”, left two) are of smaller size comparing to the wild type (WT, “+/+”, right two) age matched controls. (Panel B) Growth curve of CFTR−/− (i.e. null) vs. CFTR+/+ (WT) rabbits. CFTR null rabbits are growth retarded. (Panel C) Survival curve of CFTR null rabbits (red line) vs. CFTR+/+ (WT) rabbits. CFTR null rabbits have a 50% survival rate at approximately 40 days. (Panel D) An example of the sequencing results of CFTR gene in a CFTR heterozygous knockout rabbit (CFTR+/−) (SEQ ID NO: 1). (Panel E) An example of the sequencing results of CFTR gene in a CFTR null rabbit (CFTR−/−) (SEQ ID NO: 2). (F) Examples of mutations in CFTR null rabbits: WT (SEQ ID NO: 3 and SEQ ID NO: 7), F1#1 (SEQ ID NO: 4), F1#2 (SEQ ID NO: 5), F1#3 (SEQ ID NO: 6 and SEQ ID NO: 8).

FIG. 2 shows immunoflorescence and functional assays of CFTR expression. (Panel A) Immunocytochemistry of lungs and small intestines from CFTR+/+ and CFTR−/− rabbits. Red: CFTR; blue: nuclei. (Panel B) Short circuit current measurements of small intestines from CFTR+/+ and CFTR−/− rabbits after treated 1 μM indomethcin.

FIG. 3 shows that mucus plugs were detected in CFTR−/− rabbit airways. CFTR−/− (A-E) and CFTR+/+ (Panel F) rabbits were examined for mucus accumulation. Two of CFTR−/− rabbits were detected the mucus plugs in their trachea (Panel A), and four of CFTR−/− rabbits were found the mucus plugs in the lower airways (Panel C & Panel D). The mucus plugs in the trachea from a CFTR−/− rabbit was slided and stained with Haematoxylin and Eosin (Panel B). Lung tissue slides from CFTR−/− (Panel E) and CFTR+/+ (Panel F) rabbits were subjected to H&E stain.

FIG. 4 shows mucus accumulation and inflammatory response in the airways of CFTR−/− rabbits. H & E stain of the airways from CFTR−/− rabbits (Panel A-Panel C) and from CFTR+/+ rabbit (Panel D). al: airway lumen; car: cartilage; e: epithelial cells; g: goblet cells; boxed: neutrophils; arrows: goblet cells hyperplasia.

FIG. 5 shows that bacteria were detected in bronchioalveolar lavage fluid (BALF) from CFTR−/− rabbits. Panel A, BALF from one CF rabbit was inoculated in TSA plate. Panel B & Panel C, Gram stain of the BALF from two other CF rabbits.

FIG. 6 shows that CFTR−/− (right panels) but not WT (left panels) rabbits develop gastrointestinal obstructions due to over production of mucus. Upper: gross images of gastrointestinal (GI) tracts. Lower: HE staining of cross section of GI tracts.

FIG. 7 shows that liver fibrosis and ascites formation were found in CFTR−/− rabbits. Liver tissue slides from CFTR+/+ rabbit (Panel A), and CFTR−/− rabbit (Panel B) were stained with an antibody against collagen. Gross photo of CFTR−/− rabbit abdomen (Panel C).

FIG. 8 shows changes in gallbladder and biliary system of CFTR−/− rabbits. Panel A. gallbladders from CFTR+/+ (+/+) and CFTR−/− (−/−) littermates. H &E stain of the common ducts of CFTR+/+ (Panel B) and CFTR−/− (Panel C) and the portal triad of CFTR+/+ (Panel D) and CFTR−/− (Panel E) rabbits. Panel C1, Panel D1 and Panel E1 are zoomed images of the square box in Panel C, Panel D, and Panel E. Arrows: smooth muscle cells; box in Panel D and Panel E: portal triad; arrowheads: bile ducts.

### DEFINITIONS

To facilitate an understanding of the present disclosure, a number of terms and phrases are defined below:

As used herein, the term “animal” refers to any animal (e.g., a mammal), including, but not limited to, humans, non-human primates, rodents (e.g., mice, rats, etc.), flies, and the like.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “transgene” as used herein refers to a foreign, heterologous, or autologous gene that is placed into an organism, for example, by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “transgenic animal” refers to any animal containing a transgene.

As used herein, the term “transcriptional regulatory region” refers to the non-coding upstream regulatory sequence of a gene, also called the 5′ untranslated region (5′UTR).

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

### DETAILED DESCRIPTION OF THE DISCLOSURE

This disclosure relates to transgenic rabbit models of cystic fibrosis, and methods of using these rabbits and their derivatives.

The disclosure is described herein in reference to rabbit models of CF, which, in some embodiments, are generated by mutation, deletion, or replacement of the CFTR gene at the whole genome level or in a tissue specific manner.

The disclosure provides rabbit models of cystic fibrosis, which find use in research on the CFTR biology, the etiology of CF, as well as preclinical studies for the development and validation of approaches for diagnosis and treating CF. As is discussed further below, the rabbit models of the disclosure have been genetically modified to include one or more mutations in the endogenous CFTR gene and/or with other types of genetic changes that lead to CF related phenotypes. The genetic modifications result in the rabbits having one or more symptoms characteristic of the CF disease or condition. Rabbits exhibiting such symptoms are particularly advantageous in the development of therapeutic approaches, as candidate drugs and other approaches to treatment can be evaluated for effects on the symptoms in such rabbits.

In addition to rabbits including knock-outs or mutations in endogenous genes, the disclosure also includes transgenic rabbit models of human CF, in which one or more endogenous genes associated with the disease are knocked-out (e.g., genetically altered in such way as to inhibit the production or function of the products of these genes) and replaced with a comparable wild-type or mutated gene derived from a different animal (e.g., a human). In one example, a rabbit with its endogenous rabbit CFTR knocked-out expresses a human transgene encoding a mutated CFTR protein, such as the CFTR-F508del gene (a CFTR−/−, hCFTR-F508del rabbit). Alternatively, the human transgene may encode a normal, wild-type copy of a gene of interest (e.g., CFTR).

In some embodiments, the transgenic animals of the disclosure are made using the following strategy. Endogenous rabbit CFTR gene is disrupted by customizable genome editing enzymes, including but not limited to zinc finger nuclease (ZFN), Transcription activator-like effector nucleases (TALEN), Clustered regularly interspaced short palindromic repeats (CRISPR)/Cas9 (CRISPR-associated protein-9 nuclease), and their variants. In one example, single guide RNA (sgRNA) is designed to target the rabbit CFTR gene. The sgRNA is introduced to a rabbit cell or a rabbit embryo in the form of RNA or plasmid DNA along with Cas9 in the form of protein, mRNA or plasmid DNA. Introduction of sgRNA and Cas9 leads to insertion(s) or deletion(s) (e.g., indels) at the target locus on the rabbit genome. In some embodiments, a rabbit cell carrying indel(s) in the CFTR gene is used as the “donor cell” for nuclear transfer to produce CF rabbit. A rabbit embryo carrying indel(s) in the CFTR gene is transferred to a surrogate female to produce CF founder rabbit. Alternatively, the genome of a cell (e.g., a fetal fibroblast) from an animal of interest, such as a wild-type rabbit, is genetically modified or replaced by homologous recombination, to create a “donor cell.” The nucleus of such a genetically modified donor cell (or the entire donor cell, including the nucleus) is then transferred into a so-called “recipient cell,” such as an enucleated oocyte. After activation and, typically, a brief period of in vitro culture, the resulting embryo is implanted into a surrogate female, in which development of the embryo proceeds. Typically, the donor cell, oocyte, and surrogate female are of the same species, but the sources can be different species.

In some embodiments, after CF founder rabbits are produced and confirmed by conventional genotyping methods, they are bred with other rabbits to establish CF rabbit lines. In one example, a CF founder rabbit is bred with wild-type counterpart rabbit(s) to produce F1 generation heterozygous CF rabbits, followed by breeding F1 generation heterozygous CF rabbits with other heterozygous or homozygous CF rabbits to establish F2 generation homozygous CF rabbits.

Details of methods for making rabbits by customizable gene editing nucleases or by nuclear transfer are described below or in Du F, et al. (2009) Cloning and stem cells 11(1):131-140; Yang D, et al. (2014) J Mol Cell Biol 6(1):97-99; Song J, et al. (2016) Nature communications 7:10548. doi: 10510.11038/ncomms10548; or Yang D, et al. (2013) Journal of visualized experiments: JoVE (81):e50957.

The disclosure includes rabbits in which only one allele of a targeted gene (e.g., CFTR) is disrupted, mutated, or replaced with the other allele remaining unaffected. These animals, which are referred to herein as “heterozygous” or “hemizygous” rabbits, can be used, for example, in breeding approaches to generate homozygous mutants. These rabbits can also be used as animal models themselves.

Also included in the disclosure are homozygous mutant rabbits, in which both alleles of a target gene (e.g., CFTR) are disrupted or mutated, by the same or different mutations (or replaced with the same or different gene(s), optionally with the same or different mutations).

The transgenic rabbits of the disclosure find use, for example, in screening assay used in the development and validation of drug and other treatment methods for CF. In these methods, for example, a candidate therapeutic agent is administered to a rabbit and the impact of the agent on a feature or signs or symptom of the disease exhibited by the animal can be monitored. Optionally, the methods also involve exposure of the rabbit to environmental or other conditions known to contribute to or exacerbate the CF disease or condition. For example, in the case of CF rabbit models having impaired respiratory function, the effect of the drug on such function is assessed by measurement of standard respiratory parameters. In another example, in the case of CF rabbits exhibiting impaired digestion, due to blockage of pancreatic and/or liver ducts, the effect of a treatment on digestion is determined.

With the rabbit model of the disclosure, it is possible to test hypotheses that lead to new treatments and to evaluate potential therapies for CF lung disease. The rabbit model makes it possible to assess electrolyte transport by rabbit airway epithelia in vitro and in vivo, the volume of airway surface liquid in vitro and in vivo, the ion composition of airway surface liquid in vitro and in vivo, the airway surface liquid pH in the airway, and electrolyte transport in the small airways. It is also possible to measure respiratory mucociliary transport in vitro and in vivo. For assessing inflammation, several tests and assays can be carried out, including (but not limited to) assays of key markers of inflammation in amniotic fluid, fetal lung liquid, and bronchoalveolar lavage by using lung tissue histochemistry, large-scale gene expression profiling of pulmonary tissues, cytokine and cell assays, and proteomics. It is also possible to raise CF and non-CF rabbits in isolators under completely germ free conditions and to test for the development of pulmonary inflammation, and then selectively expose the rabbits to inflammatory stimuli including bacteria and viruses.

Although lung disease is the current main cause of mortality, patients suffer from CF disease in many other organs. Availability of a CF rabbit model allows new investigations and tests of therapeutics in the pancreas, intestine, sweat gland, liver, vas deferens, kidney, and other organs affected primarily or secondarily by CF. The screening methods of the disclosure can be carried out to test the efficacy of new compounds, combinations of new and old compounds, non-pharmaceutical treatments, and combinations of pharmaceutical and non-pharmaceutical treatments.

As described further below, CF rabbits of the disclosure have been generated and extensively characterized with respect to genotype of phenotypic characteristics. The CF rabbits have been found to share phenotypic characteristics with human CF, many of which are not also shared by murine CF models. These differences highlight the importance of the animal models of the disclosure in the development of therapeutics for human CF. Rabbit CF models of the disclosure have any one or more of the following clinical, electrophysiological, or pathological characteristics, relative to corresponding wild-type animals: (i) electrophysiological features like CF humans including, e.g., any one or more of (a) hyperpolarized baseline Vt, (b) reduction of Vt by amiloride, and (c) no CFTR or other channel activity (as measured by, e.g., perfusion of apical surface with CF-free solution and addition of isoproterenol; perfusion with ATP to activate P2Y2 receptors and Ca²⁺-activated channels; and perfusion with the CFTR inhibitor GlyH-101); (ii) meconium ileus, as characterized by, e.g., one or more of (a) obstruction in the small intestine and/or colon (e.g., near the ileocecal junction), (b) the appearance of microcolon distal to the obstruction, (c) intestinal perforation and/or peritonitis, (d) failure to pass feces or gain weight, (e) failure to eat, (f) abdominal distension, (g) bile-stained emesis, (h) proximal dilation of the small intestine, (i) meconium distension of the intestine, (j) degenerated and atrophied villi adjacent to the meconium, (k) reduced luminal diameter distal to the meconium, (l) mucinous hyperplasia (including mucoid luminal plugs) distal to the meconium, and (o) distal intestinal obstruction syndrome (DIOS); (iii) exocrine pancreatic insufficiency or abnormalities, as characterized by, e.g., one or more of (a) decreased size, (b) degenerative lobules with, e.g., increased loose adipose and myxomatous tissue, and scattered to moderate cellular inflammation, (c) diminished eosinophilic zymogen granules in residual acini, (d) variable dilation and obstruction of centroacinar spaces, ductules, and ducts with eosinophilic material plus infrequent neutrophils and macrophages mixed with cellular debris, (e) foci of mucinous metaplasia in ducts and ductules, and (f) increased redness; (iv) endocrine pancreatic abnormalities, as characterized by CF diabetes. (v) hepatic abnormalities consistent with focal biliary cirrhosis, as characterized by, e.g., any one or more of (a) mild to moderate hepatic lesions, (b) chronic cellular inflammation, (c) ductular hyperplasia, and (d) mild fibrosis; (vi) gall bladder and/or bile duct abnormalities, as characterized by, e.g., any one or more of (a) gallstones, (b) reduced size, (c) congealed bile and mucus, and (d) epithelia with diffuse mucinous changes with folds extending into the lumen; (vii) abnormalities in CF rabbits leading to male infertility; and (Viii) spontaneous airway infections.

The present disclosure contemplates the generation of transgenic animals comprising an exogenous gene or mutants and variants thereof (e.g., truncations or single nucleotide polymorphisms). In preferred embodiments, the transgenic animal displays an altered expression profile (e.g., increased or decreased presence of markers) as compared to wild-type animals. In other embodiments, transgenic animals display an altered phenotype (e.g., presence of disease) as compared to wild-type animals. Methods for analyzing the presence or absence of such phenotypes include but are not limited to, those disclosed herein.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 (1985)). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 (1976)). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. (1986)). The viral vector system used to introduce the transgene is typically a replication-defective retrovirus carrying the transgene (Jahner et al., Proc. Natl. Acad Sci. USA 82:6927 (1985)). Transfection is easily and efficiently obtained by culturing the blastomeres on a monolayer of virus-producing cells (Stewart, et al., EMBO J., 6:383 (1987)). Alternatively, infection can be performed at a later stage. Virus or virus-producing cells can be injected into the blastocoele (Jahner et al., Nature 298:623 (1982)). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra (1982)). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 (1990), and Haskell and Bowen, Mol. Reprod. Dev., 40:386 (1995)).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 (1981); Bradley et al., Nature 309:255 (1984); Gossler et al., Proc. Acad. Sci. USA 83:9065 (1986); and Robertson et al., Nature 322:445 (1986)). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 (1988)). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXPERIMENTAL

The following examples are provided in order to demonstrate and further illustrate certain preferred embodiments and aspects of the present disclosure and are not to be construed as limiting the scope thereof.

### Example 1

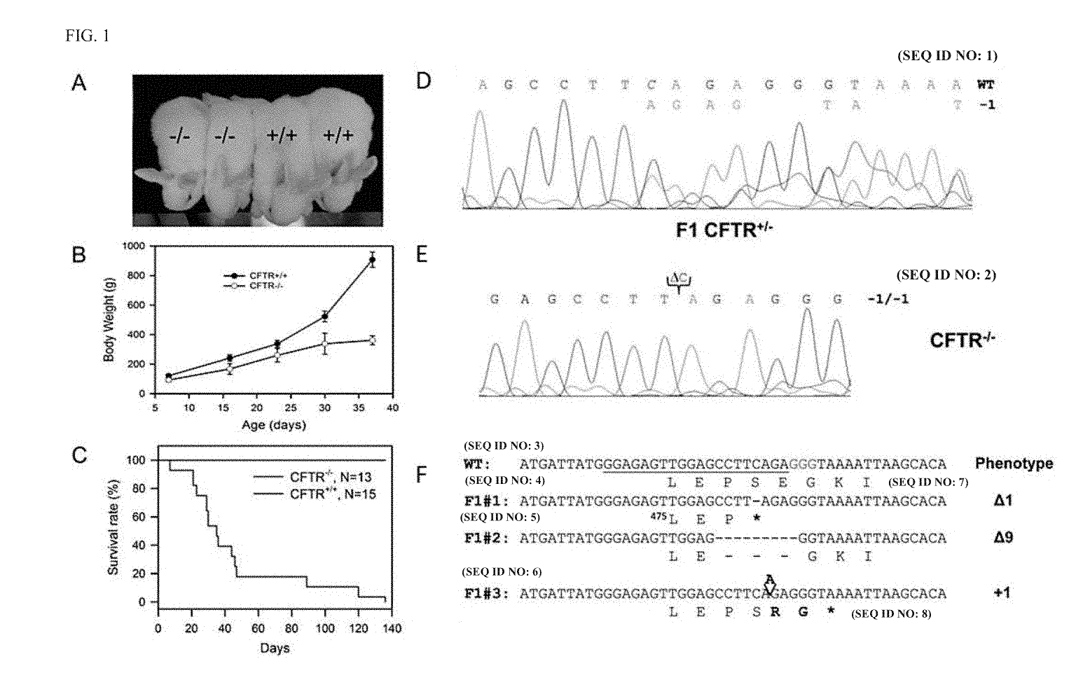

To develop a CFTR knockout model, New Zealand White rabbits were chosen because their anatomy, biochemistry, physiology, and genetics are much more similar to human than mice. CRISPR/Cas9 genome editing technology (Cong L, et al. (2013) Science) was used to generate CFTR knockout rabbits. The sgRNAs were designed based on CFTR exon 11 sequence. The targeting efficiency of five different sgRNAs (sg-01 to -05) was first tested in vitro and the different amount of particular sgRNAs and Cas9 RNA were microinjected to pronuclear stage embryos, and the sequence from the blastocysts was analyzed. Among the five sgRNAs tested, sgRNA-02, which has the targeting sequence in the exon 11, 87 bp upstream of the F508 coding site was found the most effective. Next sgRNA-02 was used for microinjection, and embryo transfer was performed. 162 embryos were transferred to 6 recipients, 11 live kits were obtained, and 3 kits were confirmed as CFTR KO F0 founders (3/11=27%). The specific indel sequences are shown in FIG. 1.

All CFTR KO F0 founders were mosaic. Upon their sexual maturation (5-6 months), F0 founders were bred with WT animals to establish the F1 generation of CFTR+/− (heterozygous) rabbits, and then F1 animals were inbred to get homozygous CFTR−/− rabbits. As showed in FIG. 1, three CF rabbit colonies were established: two of them are CFTR KO (one nucleotide deletion −1, and one nucleotide insertion, +1), and one of them is CFTR truncation (9-nucleotide deletion). The following data are from CFTR−/− rabbits with one nucleotide deletion.

1) CFTR−/− rabbits have growth retardation and a short lifespan. Three breeding pairs of CFTR+/− rabbits breeding twice gave 62 litters. Genotypes of the litters were analyzed by PCR, followed by DNA sequencing. The litters have a genotype distribution of 25.8% wild-type (n=16), 51.6% heterozygous (n=32), and 22.6% CFTR−/− homozygous rabbits (n=15). The result is similar to the expected 1:2:1 inheritance pattern. At birth, body weight was similar between CFTR−/− rabbits and their littermates (+/+); however, the first week after birth, CFTR−/− rabbits gained weight more slowly than the littermates. Upon 4 weeks after birth, CFTR−/− rabbits had significantly growth retardation (FIG. 1B). The survival rate of CFTR−/− rabbits was significantly reduced compared to that in wild-type and heterozygous littermates (FIG. 1C). The median survival time of CFTR−/− rabbits is approximately 6 wks, the time for rabbits to wean. CFTR+/− heterozygous rabbits were indistinguishable to the wt in their body weight and survival time. Of note, the CFTR−/− rabbits presented here did not receive any special care and treatment except two-day course of Golytely (twice a day) for treatment of meconium ileus. Mortality of CFTR−/− rabbits was predominantly associated with intestinal obstruction after weaning despite these rabbits who do not have meconium ileus.

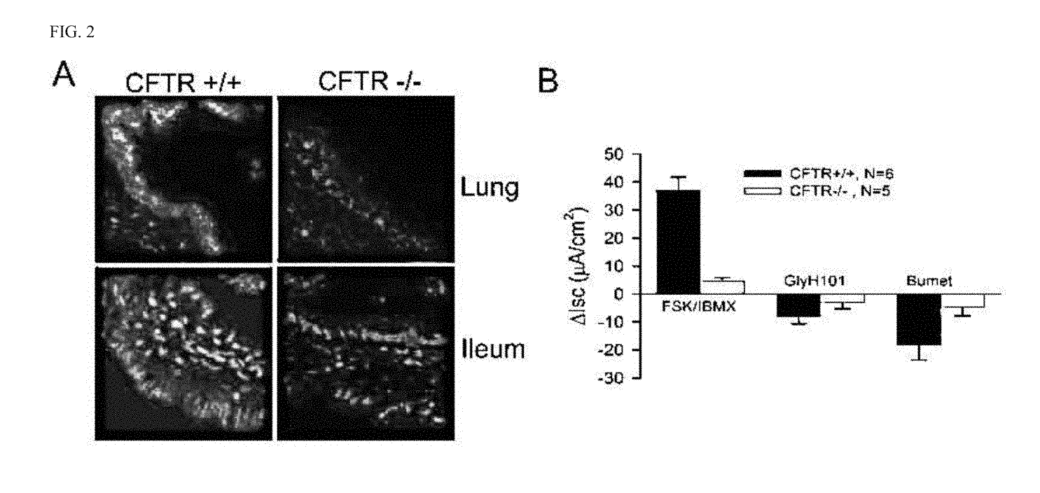

2) CFTR−/− rabbits do not have biochemical and functional CFTR expression. CFTR expression in the airways and in the small intestines of wild-type and CFTR−/− rabbits was evaluated by immunoflorescence. As shown in FIG. 2A, CFTR protein was detected on the apical membrane of normal rabbit lungs and small intestines with a lower expression level; however, no CFTR was found in CFTR−/− rabbits. Next, CFTR Cl secretion in these animals were examined. Ileum tissues from CFTR+/+ and CFTR−/− rabbits were used for CFTR functional assay. Short-circuit currents from these tissues with muscle layer on serosal side removed were measured by Ussing chamber. The freshly excised ileum tissue from CFTR+/+ rabbits exhibited a strong forskolin/IBMX-stimulated current, which was absent in CFTR−/− rabbits (p<0.001; FIG. 2B). Only a small fraction of forskolin-stimulated current was blocked by CFTR inhibitor GlyH101 at 10 μM, but bumetanide, a basolateral Na+/K+/2Cl— co-transport inhibitor, blocked more than 50% of the current, indicating that GlyH101 could not completely inhibit CFTR Cl current in the rabbit intestines. This situation was also observed in ferrets and pigs. These data demonstrated that CFTR−/− rabbits completely lost CFTR Cl channel activity.

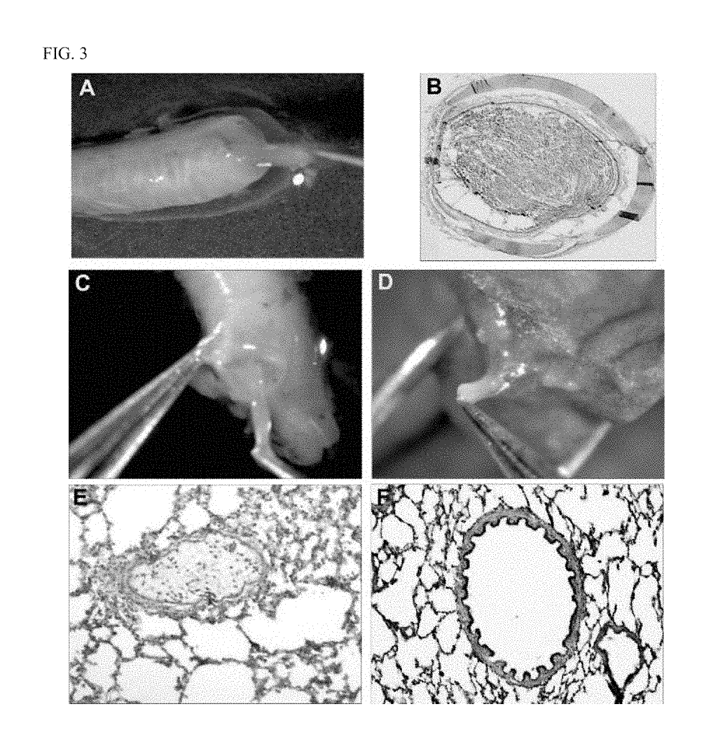

3) CFTR−/− rabbits have mucus plugs in the airways. Autopsy of CFTR−/− rabbits revealed some regions of CF rabbit lung had atelectasis and focal bronchial pneumonia (data not shown), feature characteristics of airway obstruction. Six CFTR−/− rabbits were examined for mucus accumulation in their airways. Mucus plugs were detected from trachea (FIG. 3A), bronchi (FIG. 3C). Occasionally, mucus accumulation in brochioles (FIGS. 3D & E) from CFTR−/− rabbits was also found, which are consistent with the observation in CF pigs. Mucus secretion in rabbit airways is primarily contributed by goblet cells although there is a limited number of submucosal glands in their airways. Mucus plugs detected in most CFTR−/− rabbit airways indicated that CF rabbits have defective mucociliary clearance, which is a predispose factor of lung infections in CF.

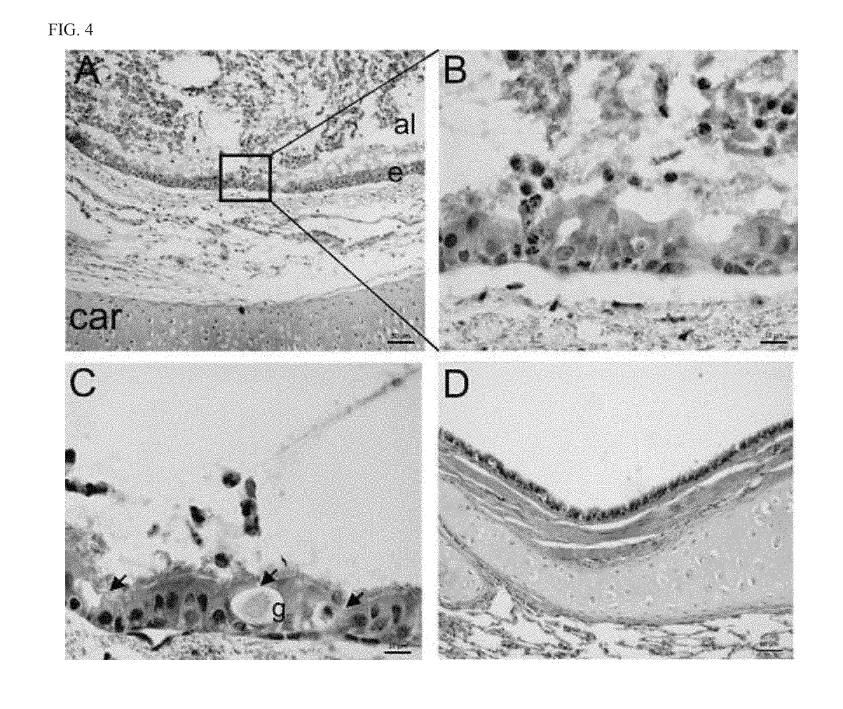

4) CFTR−/− rabbits develop mucus accumulation and inflammatory response in the airways. Primary cause of morbidity and mortality of CF patients is lung disease. In the lungs, up-regulation of mucus secretion and mucus accumulation lead to inflammatory response and infections. Compared with CFTR+/+ rabbits (FIG. 4D), airway lumen of CFTR−/− rabbits were filled with mucus, and cell debris (FIG. 4A-C), which triggered inflammatory response as demonstrated by neutrophils, and macrophages appearance in the lumen (FIG. 4A-C). Sticky mucus strings were also presented in the airways (FIG. 4C), which is a typical presentation for CF patients. Goblet cells in CFTR−/− rabbits are distended or hyperplasia and filled with mucus (FIGS. 4B&C), indicating that mucus secretion in CF rabbits is defective, which is consistent with a recent report showing dysfunction of goblet cell exocytosis leads to CF intestinal disease.

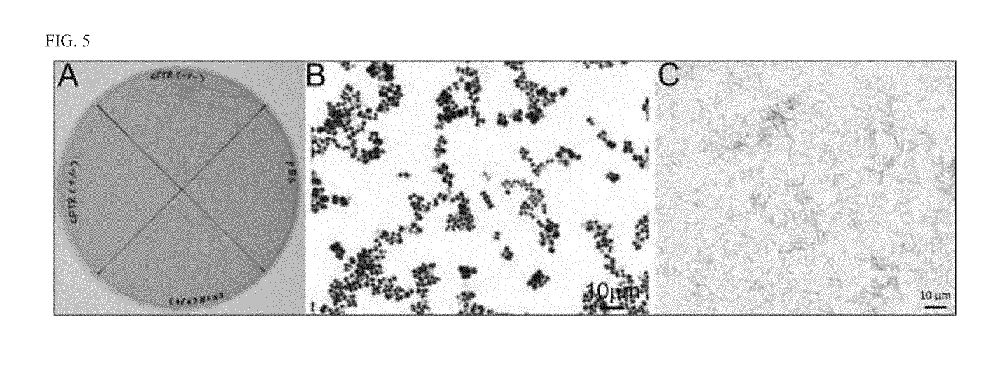

5) Bronchoalveolar lavage fluid (BALF) from CFTR−/− rabbits exhibits bacterial infections in the lungs. Bacteria infections in CFTR−/− rabbit lungs were examined. After trachea was exposed a small incision below the larynx was performed. Then a sterilized three-way stopcock connected with tubing was inserted into the trachea and secured by a surgical thread. The rabbit lungs were washed with sterile PBS three-time. Six CFTR−/− rabbits aged from 4-6 weeks were evaluated for bacterial culture of their BALF. Four out of six samples turned positive 48 hr post-incubation. The positive culture was then inoculated on Tryptone Soya Agar (TSA) plates (FIG. 5A) and subjected to Gram stain (FIGS. 5 B&C). Bacteria were found in BALF from CFTR−/− rabbits, but not detected in CFTR+/− or CFTR+/+ rabbits (FIG. 5A). Gram stain of the bacterial culture revealed the BALF from CFTR−/− rabbits contains gram positive spiral-shaped cocci (FIG. 5B) and gram negative rod-shaped bacilli (FIG. 5C). Further evaluation of the bacterial culture with 16S rRNA gene sequence analysis showed that the major bacteria founded in the BALF were Streptococcus, Staphylococcus, Stenotrophomonas, Micrococcaceae, and Enterobacteriaceae. One CFTR−/− rabbit was detected Pseudomonas infection in the lungs. These data demonstrated that CF rabbits have spontaneous lung infections. These data strongly suggest that the lungs from CF rabbits have an impaired ability to eradicate bacteria, which is consistent with data from humans showing that many different bacteria can infect CF lungs, especially early in the course of disease.

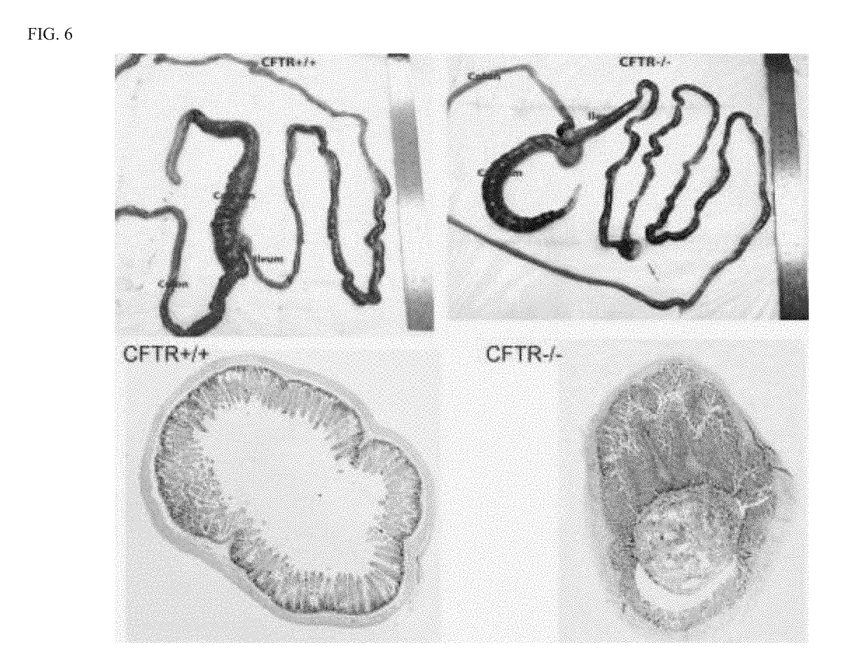

6) CFTR−/− rabbits do not have significant meconium ileus, but have gut obstruction after their weaning. Meconium ileus occurs 100% of CF pigs and 75-90% of CF ferrets, which is a clinical presentation in CF patients at prevalence of about 20%. Unlike CF pigs and CF ferrets, meconium ileus was rarely observed in CFTR−/− rabbits within the first month after birth because rabbits have a large functional cecum although the animal eventually developed distal intestine obstruction. Therefore, CF rabbits have a relative sufficient window time to study the pathogenesis of the disease without any special care and expensive treatments. As showed in FIG. 6 right upper panel, the 5-week old CF rabbit has the feces obstruction in the cecum and the intestines, but not in CFTR+/+ animal shown in FIG. 6 left upper panel. H &E stain of CFTR−/− rabbit revealed a massive mucus plug obstruction in the intestines lumen (FIG. 6 lower panels).

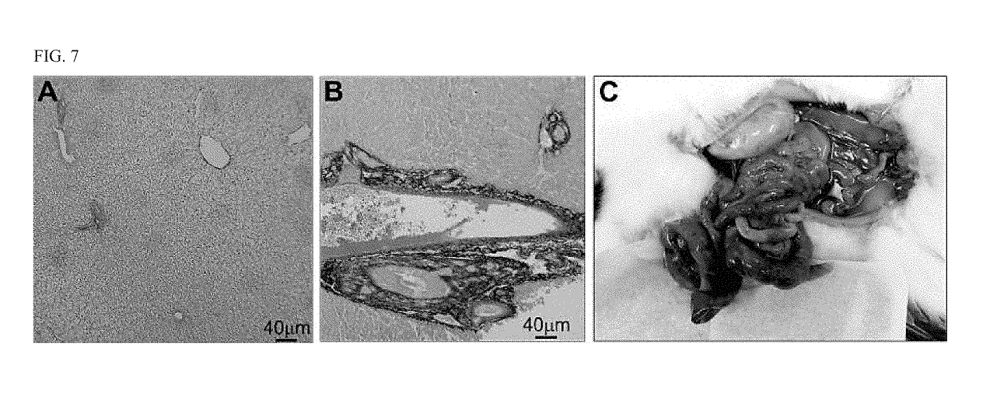

7) CFTR−/− rabbits have liver fibrosis, ascites, and micro-gallbladder with associated pathology. Liver disease occurs one third of CF patients and is the second cause of death in the patients although the most common one is lung disease. In the hepatobiliary system, loss of CFTR leads to focal biliary cirrhosis, portal hypertension, micro-gallbladder, and common bile duct stenosis. CFTR−/− rabbits were examined. Although gross anatomy did not show visible changes between CFTR+/+ and CFTR−/− rabbits, CFTR−/− rabbits have a strong deposition of collagen in the biliary triad (FIG. 7B) and visible ascites (FIG. 7C) in spite of the fact that the liver enzymes in CFTR−/− rabbits were only mildly elevated (data not shown). The liver fibrosis was detected approximately 15% (2/13) whereas ascites was found more than 50% (7/13) of CF rabbits.

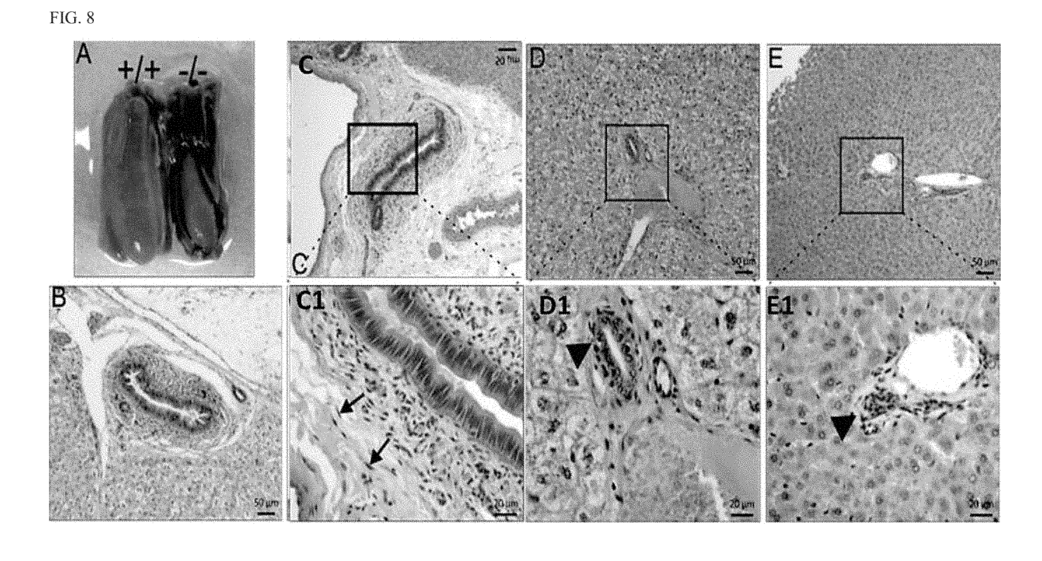

Among the gastrointestinal manifestations observed in CF patients, gallbladder abnormalities occur frequently. These include a high prevalence of nonfunctioning gallbladders (30%), micro-gallbladders (8-30%) and gallstones (4-30%). Approximately 50% of CF rabbits have microgallbladder (FIG. 8A). Compared with wt rabbits (FIG. 8B), the common bile duct of CF rabbits has fibrosis and chronic inflammation (FIG. 8C).

The epithelial cells lining the bile duct are disoriented and the ducts are stenosis in CF rabbits (FIG. 8E), but are not in wt rabbits (FIG. 8D). These data indicated that CF rabbits have obstruction of intrahepatic bile ducts, causing focal biliary cirrhosis, inflammation, and fibrosis, presumably because of overproduction of viscous mucus.

In summary, the CFTR knockout rabbits generated recapitulate many clinical features observed in CF patients. The relative lower maintenance cost and short reproduction period make rabbit CF model more attractive.

All publications, patents, patent applications and accession numbers mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. A transgenic rabbit comprising a genome that comprises:
(i) one or more mutations in one or both alleles of the rabbit genome resulting in altered rabbit cystic fibrosis transmembrane regulator (CFTR) gene expression and one or more signs or symptoms of cystic fibrosis (CF) in the rabbit; and
(ii) a CFTR transgene under the control of a promoter resulting in whole body or tissue-specific CFTR transgene expression in the rabbit.

2. The transgenic rabbit of claim 1, wherein one or both allels of said CFTR gene of said rabbit are knocked out.

3. The transgenic rabbit of claim 1, wherein one or both alleles of said CFTR genes are replaced with mutant CFTR genes.

4. The trangenic rabbit of claim 3, wherein said mutant CFTR gene comprises a CFTR-F508del mutation.

5. The transgenic rabbit of claim 1, wherein said exogenous CFTR gene is under the control of an intestinal specific promoter.

6. The transgenic rabbit of claim 5, wherein said intestinal cell-specific promoter is an intestinal fatty acid binding protein (iFABP) promoter.

7. The transgenic rabbit of claim 1, wherein said exogenous CFTR gene is a mutant human or rabbit CFTR gene.

8. The transgenic rabbit of claim 7, wherein said mutant human CFTR gene comprises a deletion of F508.

9. The transgenic rabbit of claim 1, wherein said rabbit has one or more phenotypes selected from the group consisting of (a) an electrophysiological phenotype like that of human cystic fibrosis, (b) pancreatic insufficiency or abnormalities, (c) hepatic abnormalities, (d) gall bladder and/or bile duct abnormalities, (e) sweat gland abnormalities, (f) kidney abnormalities, (g) cystic fibrosis related diabetes, (i) bilateral congenital absence of the vas deferens leading to male infertility, (j) tracheal abnormalities, (k) cystic fibrosis lung disease; and (l) cystic fibrosis eye disease.

10. A method of determining whether a candidate therapeutic approach can be used in the treatment of cystic fibrosis, the method comprising:
a) carrying out said candidate therapeutic approach on a transgenic rabbit of claim 1; and
b) and monitoring the rabbit for one or more symptoms of cystic fibrosis.

11. The method of claim 10, wherein detection of improvement in one or more symptoms of cystic fibrosis in said rabbit indicates the identification of a therapeutic approach for the treatment of cystic fibrosis.

12. The method of claim 10, wherein the candidate therapeutic approach comprises administration of a candidate therapeutic agent.

13. The method of claim 11, wherein the symptom of cystic fibrosis is monitored in one or more organs selected from the group consisting of tracheal, lung, pancreas, liver, kidney, and vas deferens.

14. An isolated cell or tissue of a rabbit of claim 1.

15. The isolated cell or tissue of claim 14, wherein said cell or tissue is selected from the group consisting of tracheal, lung, pancreas, liver, kidney, and vas deferens origin.


SEQUENCE LISTING (8 sequences)

SEQ ID NO: 1 - DNA, 18 bp, Oryctolagus cuniculus
agccttcagagggtaaaa

SEQ ID NO: 2 - DNA, 13 bp, Oryctolagus cuniculus
gagccttagaggg

SEQ ID NO: 3 - DNA, 46 bp, Oryctolagus cuniculus
atgattatgggagagttggagccttcagagggtaaaattaagcaca

SEQ ID NO: 4 - DNA, 45 bp, Oryctolagus cuniculus
atgattatgggagagttggagccttagagggtaaaattaagcaca

SEQ ID NO: 5 - DNA, 36 bp, Oryctolagus cuniculus
atgattatgggagagttggagggtaaaataagcaca

SEQ ID NO: 6 - DNA, 46 bp, Oryctolagus cuniculus; identical to SEQ ID NO: 3

SEQ ID NO: 7 - protein, 8 aa, Oryctolagus cuniculus
LEPSEGKI

SEQ ID NO: 8 - protein, 6 aa, Oryctolagus cuniculus
LEPSRG